(19) United States
(12) Patent Application Publication
(10) Pub. No.: US 2006/0212115 A1
(43) Pub. Date: Sep. 21, 2006

(54) Intracorneal ring and procedure to place same

(76) Inventor: Arturo Maldonado Bas, Cordoba (AR)

Correspondence Address: BACHMAN & LAPOINTE, P.C., 900 CHAPEL STREET, SUITE 1201, NEW HAVEN, CT, 06510, US

(21) Appl. No.: 11/378,814

(22) Filed: Mar. 17, 2006

(30) Foreign Application Priority Data
Mar. 18, 2005 (AR) P20050101063

(51) Int. Cl. A61F 2/14

(52) U.S. Cl. 623/5.12

(57) ABSTRACT

The intracorneal ring is made up of a suitable material deformable with compression and with an appropriate molecular memory so that, being at rest, it can take the shape of a ring. The ring has an outside diameter between 5 and 7 mm, a wall thickness between 0.25 and 0.4 mm, and an inner diameter of about 4.5 to 6.2 mm. When dealing with the keratoconus treatment, the ring is placed in a concentric position with respect to the cone apex and inside the thickest area of the cornea. In the astigmatism correction, it is placed in a pocket done in the corneal stroma, thus behaving as a curvature modifier of the cornea anterior face.

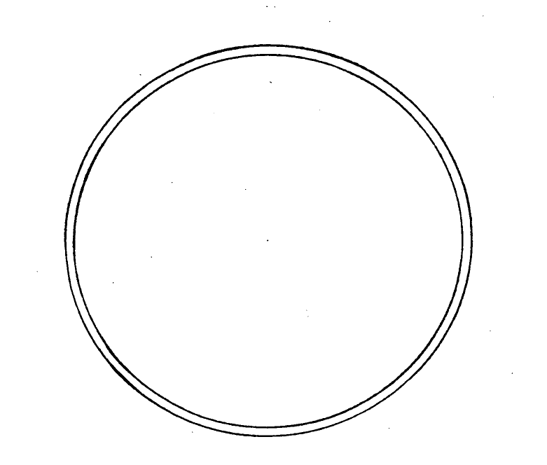

### BACKGROUND OF THE INVENTION

(1) Field of the Invention

This invention relates to an intracorneal ring and the procedure to position it. In order to make this invention comprehensible and to be able to put it into practice without difficulties, there will follow a precise description of one preferred manner of accomplishment in the subsequent paragraphs. Reference will be made to the drawings that illustrate and accompany this description as examples of said preferred way of carrying this procedure, but neither the description nor the diagrams should be considered as limiting the invention. The components explained may be selected by experts in the subject from among several equivalents, without this implying a deviation from the principles established in the present documentation.

(2) Prior Art

The intracorneal rings used in the state of the technique, are as a matter of fact rigid semicircular segments made of polymethylmethacrylate which are placed in the corneal stroma in order to modify the curvature of the cornea interior face. Therefore, they are not “rings” in the real sense of the word but they are actually semicircular segments.

The first research about this subject was carried out by Gabriel Simón in the Vascom Palmer several years ago.

The aforementioned designed a spiral shaped instrument that allowed him to make an incision within the cornea up to the intended depth. For example, over a 500 micron depth, a 300 micron deep incision could be made.

In this way, first Simon and then those who followed him, were able to carve a channel making good use of the cornea characteristics since it displays its lamellas parallel and outstretched between both ends making it easier to find the plane to make the incision.

Ferrara rings are being used at present but, as it was said before, they are as a matter of fact two segments of about one hundred and eighty degrees in circle each of them.

Both segments can be combined in such a way that they may have the same or different thickness and for example, by using a thick segment in the lower part and a thinner one in the superior half, the keratoconus can be corrected.

As Ferrara rings do not have continuity, they do not obviously take the shape of a complete circle.

Their function is to achieve a stretching of the anterior surface of the cornea. That is the reason why each of the opposing segments needs to have thickness and volume enough to bear the necessary rigidity. Said rigidity allows to transfer the right tenseness to the cornea to be able to achieve stretching.

The idea to put two semicircles comes as a result of the practice itself, since in doing the semicircular dissection using the spiral shaped instrument designed by Gabriel Simón, it is impossible to place a full ring.

Therefore, when using the abovementioned instrument it is suggested to carry out a cutting three millimeters from the visual axis and to the desired depth so that, with the same instrument a dissection can be made following the parallel lamellas of the cornea to the depth selected with the cut.

This allows to make a semicircular canal in each of the two halves so that later the Ferrara rings can be introduced.

If instead of placing two semicircles as it is done in the state of the technique they are replaced by a full ring of the same thickness and volume, the material resistance would cause quite a higher tension. Being this tension unnecessary, such ring could be considerably reduced thus lessening significantly the patient discomfort and reducing adaptation period.

### SUMMARY OF THE INVENTION

It is therefore the purpose of this invention to replace Ferrara rings, in fact the ring segments, for a complete ring of a lesser thickness and volume.

As a consequence derived from the ring shape being disclosed, the operating technique will have to be adapted.

The invention being disclosed consists of a ring adapted in a suitable material such as a polymethylmethacrylate or similar, appropriate to be placed in an intracorneal position. Likewise, the positioning procedure is hereinafter disclosed.

### BRIEF DESCRIPTION OF THE DRAWINGS

With the intention of better understanding the description and functioning being disclosed, the FIGURE illustrates an outlined intracorneal ring.

### DETAILED DESCRIPTION OF THE PREFERRED EMBODIMENT(S)

Having the different components that explain the nature of the invention been established, this description is now complemented with the functional and operational explanation of its parts and the result they bring about.

The present invention has proceeded to replace the two semicircular segments used to tighten the cornea by a single ring. This ring, being placed in the cornea peripheral area, has a circular anatomic integrity that allows it to exert a complete symmetrical force. Said force, together with the molecular memory of the ring material, lets the ring thickness to be substantially reduced, thus increasing its diameter to attain a clinical corneal flattening in order to correct the astigmatism and myopia caused by the keratoconus.

This proposed ring is made up of a biocompatible heterologous material such as a polymethylmethacrylate or similar.

Laws enunciated by Professor Barraquer establish that the tissue added to the corneal periphery corrects myopia whereas tissue added in the centre does the same with the hypermetropia.

Since this ring being hereby disclosed is added to the corneal stroma and thanks to said addition the corneal stroma thickness is increased, it is inferred that said ring complies with Barraquer laws and will be correcting the myopia.

As it was previously mentioned, among the advantages of placing a complete ring instead of two semicircular segments, there exists the benefit of peripheral thickness increase that will be interpreted as a tissue addition and this, according to the abovementioned Barraquer laws, will allow myopia to be corrected.

Given that it is a full ring, the tenseness it produces to the compression turns out to be homogeneous and therefore, as a result of this tenseness the cornea anterior face can be tensed and flattened and in this manner astigmatism can be corrected.

As a result of the thickness reduction of the ring wall, the strange body action is reduced. This brings about the lessening of the discomfort and shortens the patient recovery period turning down at the same time the extrusion danger, a circumstance which has sometimes occurred with the inclusion of the Ferrara rings.

In accordance with the present invention, it has been estimated that the intracorneal ring must be between 5 and 7 mm outside diameter, consequently tests have been carried out with 6 mm outside diameter rings bearing 0.3 mm wall thickness to allow 5.4 inner diameter.

In like manner tests have been accomplished with a 6.5 mm external diameter ring; 0.35 mm wall thickness and 5.8 mm inner diameter.

It has been estimated that, for those cases in which the keratoconus is tried to be rectified, it is proper to place the ring in a concentric position with respect to the cone apex searching for a healthier and thus thicker area of the cornea, suitable to prevent the extrusion found with the Ferrara rings.

On the other hand, if the ring is small it will be able to flatten the central area more easily, mainly in a 3.5 mm area which is the one most specifically used in vision.

The ring location that is being disclosed turns out to be different from the Ferrara rings. Certainly, the Ferrara ring is introduced in a channel carved with a spiral shaped instrument by means of an incision done about 2.5 mm from the visual axis.

On the other hand, in order to be able to locate the proposed intracorneal ring, it is required that a procedure be followed to ascertain corneal thickness through the use of a pachymetric map and the location of the keratoconus by means of the mydriasis retro lighting.

In another step, making use of the already described Melles dissection used to do the deep laminated keratoplasties of the anterior segment, a dissection is carried out from the limb and through an incision between 5 and 5.5 mm with a depth not exceeding 80% of the corneal apex pachymetry.

From this limbal incision, the dissection is started with a crescent bevel up thus forming a pocket in the cornea following the parallel cut lamellas until the central area is reached.

In another step, using a dissector with a concave lower surface (like the ones originally designed by Barraquer, then by Melles and finally by Mark Terri) a mm diameter pocket is then dissected which will leave room for a ring between 6 or 6.5 mm.

In a previous step and in order to both achieve a diameter as accurate as possible and not do an exaggerate dissection of the corneal stroma, it is convenient to mark the visual axis before starting the surgery and the keratoconus apex that is usually downward the referred visual axis.

It will be then over one of these two axis that, centering with the cone or the pupillary axis, an external mark is done with gentian violet over the corneal epithelium to work as the guide over which the dissection is adjusted accurately to the area where the pocket must be dissected.

Once the dissection has been done, and in a next step, the intracorneal ring is held with one of those pincers used to bend flexible intraocular lens and it is smoothly pressed to warp it into an oval shape in order to pass the 5.5 mm incision.

Once the ring has been placed in the pocket, in the following step the pincers are opened so that the ring regains its circular shape thanks to its molecular memory and it is unfurled inside the corneal stroma.

In another step and with an appropriate instrument such as a Becker pusher, it is softly thrust until placed in the desired position.

As last step, the incision is sutured with one or two stitches.

In order to select the size of the intracorneal ring to be used it is necessary to take into account the corneal thickness, that is to say the pachymetry measured in all the surface and the degree of ectasia.

These two parameters are the ones that, combined with the particular case, will determine the measures of the best appropriate ring.

The foregoing depicts one of the possible ways to put the invention into effect and the manner in which it functions. The documentation hereinafter is complemented with the synthesis of the invention contained in the claim clauses appended below.

Having described and determined the nature of the invention, its scope and the way in which its fundamental concept can be put into practice, the following is declared as invention and exclusive property:

1. Intracorneal ring made up of a biocompatible material comprising a suitable material deformable with compression and with an appropriate molecular memory so that while at rest the material can take the shape of a ring, said ring having an outside diameter between 5 and 7 mm, a wall thickness between 0.25 and 0.4 mm, and an inner diameter about 4.5 to 6.2 mm, wherein, in keratoconus treatment, said ring is placed in a concentric position with respect to a cone apex and in a thickest area of the cornea, and wherein in astigmatism correction, said ring is placed in a pocket done in a corneal stroma, thus behaving as a curvature modifier of a cornea anterior face.

2. The intracorneal ring according to claim 1, wherein said ring is made of an heterologous and biocompatible material.

3. The intracorneal ring according to claim 1, wherein said ring is made of polymethylmethacrylate.

4. A procedure to place an intracorneal ring made up of a biocompatible material comprising a suitable material deformable with compression and with an appropriate molecular memory so that while at rest the material can take the shape of a ring, said ring having an outside diameter between 5 and 7 mm, a wall thickness between 0.25 and 0.4 mm, and an inner diameter about 4.5 to 6.2 mm, implying an incision and a subsequent suture, comprising: a first step to mark a visual axis and an apex of a keratoconus and a selection of one of those axes so that, centering with a cone or a papillary axis, an external mark can be made with gentian violet over a corneal epithelium as a guide to determine a dissection area of a pocket to hold the ring; a second step to determine a corneal thickness and keratoconus location; a third step to make a limbal incision between 5 and 5.5 mm with a depth 80% less that a corneal apex pachometry, dissection and formation of a pocket in the cornea following lamellas of parallel faces until reaching a central area; a fourth step to dissect the pocket formed in the previous step; a fifth step in which, in a first stage the intracorneal ring is held with pincers appropriate to blend flexible intraocular lens, in a second stage the ring is pressed and deformed in order to get it oval shaped, in a third stage the ring is introduced in the dissected pocket and in a fourth stage the pincers are opened so that the ring regaining its original shape can unfold inside the corneal stroma and in a sixth step, the intracorneal ring is placed in a desired position in relation to pocket borders.

5. The procedure according to claim 4, wherein in the second step the corneal thickness is determined by means of a pachometric map and the location of the keratoconus by means of mydriasis retro lighting.

6. The procedure according to claim 4, wherein in the third step the dissection is carried out with a crescent bevel up.

7. The procedure according to claim 4, wherein in the fourth step a dissector with a concave inferior surface is used in order to dissect a pocket of a slightly bigger diameter than the intracorneal ring to be placed.

8. The procedure according to claim 4, wherein in the sixth step the intracorneal ring is placed in the desired position while thrusting the ring softly with an instrument.
